(19) United States
(12) Patent Application Publication
(10) Pub. No.: US 2010/0048983 A1
(43) Pub. Date: Feb. 25, 2010

(54) Multipath Stimulation Hearing Systems

(75) Inventors: Geoffrey R. Ball, Axams (AT); Claude Jolly, Innsbruck (AT)

Correspondence Address: Sunstein Kann Murphy & Timbers LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/545,325

(22) Filed: Aug. 21, 2009

Related U.S. Application Data
(60) Provisional application No. 61/090,758, filed on Aug. 21, 2008.

(51) Int. Cl. A61F 11/04; A61N 1/36

(52) U.S. Cl. 600/25; 607/57

(57) ABSTRACT

A prosthetic hearing system is described that provides multi-path stimulation of the patient auditory system. A mechanical stimulation component applies mechanical stimulation signals to cerebral tissue such as the dura mater, cerebrospinal fluid, vestibular structures, etc. using multiple separate mechanical stimulation channels. And an electrical stimulation component provides electrical stimulation of auditory neural tissue of the patient user.

This application claims priority from U.S. Provisional Application 61/090,758, filed Aug. 21, 2008, which is incorporated herein by reference.

### FIELD OF THE INVENTION

The present invention relates to medical implants, and more specifically to auditory prosthetic implant systems that produce mechanical and electrical stimulation.

### BACKGROUND ART

The seemingly simple act of hearing can easily be taken for granted. Although it may seem that we exert no effort to hear the sounds around us, from a physiologic standpoint, hearing is an awesome process. The hearing mechanism involves a complex system of levers, membranes, fluid reservoirs, neurons and hair cells which must all work together in order to deliver nervous stimuli to the brain where this information is compiled into the higher level perception we think of as sound.

In this complicated mix of acoustic, mechanical and neurological systems, much can go wrong. It is estimated that one out of every ten people suffers from some form of hearing loss. Surprisingly, many patients who suffer from hearing loss take no action to treat the condition. In many ways hearing is becoming more important as we move toward an information based society, but unfortunately for the hearing impaired, success in many professional and social situations may be becoming more dependent on effective hearing.

Those in the field of hearing science are well aware of advances that are being made to help combat hearing loss and further the scientific understanding of the hearing processes. Several ongoing projects have been instrumental in demonstrating the potential for advanced devices to help the hearing impaired. Although conventional acoustic hearing devices have been helpful to many of the hearing impaired, most of the world wide impaired population, for whatever reason, forego their use. Hopefully, as technical advancements are made and alternative devices begin to appear, more hearing impaired patients will get the help they need.

The first known hearing prosthetic device appeared in Roman times and used a hollow dome “catch” that probably provided about 15-20 decibels of sound amplification for the user. Ear trumpets and conversation tubes were widely available in the 1700's and 1800's, and the first electronic hearing aids debuted in the early 1900's.

The development of the transistor lead to smaller more power efficient hearing aids which began to appear in the 1950's. In the 1960's and 1970's, there was a period of accelerated development in which hearing device companies and product lines began to rapidly multiply. Measurement standards for prescribing prosthetic hearing devices, patient hearing evaluation, and manufacturing standards for hearing devices became more established, and audiologists were able to advance device technology, continue hearing research, and institute improvements in hearing device measurement and fitting technology. Audiologic advancements in hearing assessment and diagnosis of hearing disorders translated into better diagnosis and treatment for the hearing impaired. State regulation of the dispensing industry through licensing and certification programs also was developed to insure quality of hearing aid dispensing practices.

At present, a hearing impaired patient has a wide variety of prosthetic hearing devices to choose between. Current devices have improved signal processing circuits and enhanced fitting parameters that allow the device to be customized to the patient's individual hearing loss (i.e., similar to an eye glass prescription, one size does not fit all). New devices can be located entirely in the patient's ear canal and are cosmetically superior to the large bulky devices of years past. Many manufacturers participate in the hearing marketplace which is a sizable 3 billion dollar worldwide market.

A normal ear transmits sounds as shown in FIG. 1 through the outer ear 101 to the tympanic membrane (eardrum) 102, which moves the bones of the middle ear 103 (malleus, incus, and stapes), which in turn vibrate the oval window and round window openings of the cochlea 104. The cochlea 104 includes an upper channel known as the scala vestibuli and a lower channel known as the scala tympani which are connected by the cochlear duct. In response to received sounds transmitted by the middle ear 103, the fluid filled scala vestibuli and scala tympani function as a transducer to transmit waves to fine hair receptor cells within the cochlea 104 that generate electric pulses that are transmitted to the cochlear nerve 113, and ultimately to the brain.

The vibratory structures of the ear include the tympanic membrane 102, the middle ear 103 (the ossicles—malleus, incus, and stapes, the oval window, and the round window), and the cochlea 104. Each of these vibrates to some degree when a person with normal hearing hears sound. But hearing loss may be evidenced reduced or no vibration in one or more of these structures. For example, the ossicles in the middle ear 103 may lack the resiliency needed to increase the force of sound vibrations enough to adequately stimulate the receptor cells in the cochlea 104. Or the ossicles can be broken so that they do not conduct sound vibrations to the oval window and/or the round window of the cochlea 104.

Prostheses for ossicular reconstruction are sometimes implanted in patients who have partially or completely broken ossicles. These prostheses are designed to fit snugly between the tympanic membrane 102 and the oval window or stapes. The close fit holds the prosthesis in place (although gelfoam is sometimes packed into the middle ear 103 to guard against loosening). Although these prostheses provide a mechanism by which vibrations may be conducted through the middle ear to the oval window of the inner ear, additional devices are frequently necessary to ensure that vibrations are delivered to the inner ear with sufficient force to produce high quality sound perception.

With conventional hearing aids, a microphone detects sound which is amplified and transmitted in the form of acoustical energy by a speaker or another type of transducer into the middle ear 103 by way of the tympanic membrane 102. Interaction between the microphone and the speaker can sometimes cause an annoying and painful a high-pitched feedback whistle. The amplified sound produced by conventional hearing aids also normally includes a significant amount of distortion.

Efforts have been made to eliminate the feedback and distortion problems, yielding devices which convert sound waves into electromagnetic fields having the same frequencies as the sound waves. A coil winding is held stationary by attachment to a non-vibrating structure within the middle ear 103 and microphone signal current is delivered to the coil winding to generate an electromagnetic field. A magnet is attached to an ossicle within the middle ear 103 so that the magnetic field of the magnet interacts with the magnetic field of the coil. The magnet vibrates in response to the interaction of the magnetic fields, causing vibration of the bones of the middle ear 103. See U.S. Pat. No. 6,190,305, which is incorporated herein by reference.

Existing electromagnetic transducers can present some problems. Many are installed using complex surgical procedures which present the usual risks associated with major surgery and which also require disarticulating (disconnecting) one or more of the bones of the middle ear 103. Disarticulation deprives the patient of any residual hearing he or she may have had prior to surgery, placing the patient in a worsened position if the implanted device is later found to be ineffective in improving the patient's hearing.

Existing devices also are unable to produce substantially linear and high quality vibrations directly to the cochlea 104. Direct mechanical stimulation of the cochlea 104 with a hi-fidelity transducer bypasses some signal interference. But previous devices have not been closely coupled with the cochlear fluid so that the resulting sound often is significantly distorted because the vibrations conducted to the cochlea 104 do not accurately correspond to the sound waves detected by the microphone.

Cochlear implants utilize electrical signal to directly stimulate the cochlea 104 via electrical stimulation. These devices have been utilized for over 30 years with excellent success and are generally appropriate for patients with severe or profound hearing loss who cannot make use of conventional acoustic hearing aids, bone anchored devices, middle ear implants or surgical reconstruction or a combination thereof.

FIG. 1 also shows some components of a typical cochlear implant system. A typical system may include an external microphone that provides an audio signal input to an external signal processing stage 111 where various signal processing schemes can be implemented. The processed signal is then converted into a digital data format, such as a sequence of data frames, for transmission into by an external transmitter coil 107 into implant module 108. Besides extracting the audio information, the implant module 108 also performs additional signal processing such as error correction, pulse formation, etc., and produces a stimulation pattern (based on the extracted audio information) that is sent through connected wires 109 to an implanted electrode carrier 110. Typically, this electrode carrier 110 includes multiple electrodes on its surface that provide selective stimulation of the cochlea 104.

Existing cochlear implant systems need to deliver electrical power from outside the body through the skin to satisfy the power requirements of the implanted portion of the system. FIG. 1 shows a typical arrangement based on inductive coupling through the skin to transfer both the required electrical power and the processed audio information. As shown in FIG. 1, the external transmitter coil 107 is placed on the skin adjacent to the subcutaneous implant module 108 which contains a corresponding receiver coil. Often, a holding magnet in the external transmitter coil 107 interacts with a corresponding implant magnet in the implant module 108. This arrangement inductively couples a radio frequency (rf) electrical signal to the implant module 108. The implant module 108 is able to extract from the rf signal both the audio information for the implanted portion of the system and a power component to power the implanted system.

In most prior systems, the external components generally have been held in separate housings so that the external transmitter coil 107 would not be in the same physical housing as the power source or the external signal processing stage 111. The various different physical components would generally be connected by hard wire, although some systems used wireless links between separate external components. A few systems have been proposed in which all of the external components such as an external processor and a rechargeable battery could be placed within a single housing. See U.S. Patent Publication 20080002834 (Hochmair) and U.S. Patent Publication 20070053534 (Kiratzidis), which are incorporated herein by reference.

### SUMMARY OF THE INVENTION

Embodiments of the present invention include a prosthetic hearing system that provides multi-path stimulation of the patient auditory system. A mechanical stimulation component applies mechanical stimulation signals to cerebral tissue such as the dura mater, cerebrospinal fluid, inner ear vestibular structures, etc. using multiple separate mechanical stimulation channels. And an electrical stimulation component provides electrical stimulation of auditory neural tissue such as the cochlear nerve and/or the brainstem.

The mechanical stimulation component may include a floating mass transducer, a vibrating element transducer, a balanced armature transducer, an inertial drive transducer, a rotating transducer, a rotational magnet and armature transducer, or a vibrating magnet with an associated coil (e.g., with a secondary magnet component that cooperates with a remotely located primary vibrating magnet component). The electrical stimulation component may include one or more stimulation electrodes with one or more electrode contacts for neural stimulation of the auditory neural tissue. There may also be a secondary electrical ground unit for the electrodes.

A specific embodiment may also include a main implant housing having a top surface nearest the skin of the patient and a bottom surface associated with the mechanical stimulation component. The main implant housing may be directly coupled to the cerebral tissue to provide at least one of the mechanical stimulation channels. In such an embodiment, the mechanical stimulation component may also include a vibrating coupling rod functionally coupled to mechanically stimulate the cerebral tissue. In some embodiments, the main implant housing may provide multiple mechanical stimulation channels.

There may be an implant receiver coil within the main implant housing for receiving an externally generated implant signal. In addition or alternatively, there may also be an implant signal processor for processing the implant signal. A sound sensing arrangement may generate the implant signal using a microphone arrangement such as an external omni-directional microphone, multiple external sensing microphones, or an implanted microphone for sensing sound. The entire system may be implantable within a patient and may include a hybrid operating mode which communicates with external devices. The implant signal processor may process the implant signal based on frequency content such that a first band of frequencies is associated with the electrical stimulation component and a second band of frequencies is associated with the mechanical stimulation component. For example, the second band of frequencies for the mechanical stimulation component may possess a peak resonance audio frequency between 0.25 and 3.5 kHz.

The main implant housing may be constructed of a biocompatible material including one or more of medical grade titanium, ceramic, silicone, and acrylic. The mechanical stimulation component may be located within or outside the main implant housing. The mechanical stimulation component may include a connectorized lead for connecting the mechanical stimulation component to the main implant housing. Similarly, the electrical stimulation component also may include a connectorized lead for connecting a portion of the electrical stimulation component to the main implant housing.

The system may include an implanted sensing microphone connected to the main implant housing for monitoring performance of the mechanical stimulation component. Similarly, there may be a performance monitor for monitoring performance of the mechanical stimulation component. The electrical stimulation component may be mechanically isolated from the vibrations of the mechanical stimulation component. The electrical stimulation component may provide one or more of the mechanical stimulation channels. The system may also include a fluid-filled catheter tube for providing one of the mechanical stimulation channels. There may be an electrical module housing associated with the electrical stimulation component and a separate mechanical stimulation module associated with the mechanical stimulation component. And either or both module housings may provide mechanical stimulation channels.

The auditory neural tissue stimulated by the electrical stimulation component may include cochlear nerve tissue and/or brainstem tissue. The mechanical stimulation component may include a vibrating plate in contact with the cerebral tissue. The cerebral tissue stimulated by the mechanical stimulation channels may include dura mater tissue, cerebrospinal fluid, inner ear tissue such as vestibular tissue, and/or bone tissue such as a temporal bone or skull bone. The vibrations may have a displacement less than or equal to 100 microns peak to peak.

The electrical stimulation component and the mechanical stimulation component may be synchronously operable to operate in parallel at the same time, and/or alternately operable to operate serially one at the same time,

The mechanical stimulation component may be fixed into position with respect to the auditory tissue by one or more screws, a silicone elastomer membrane, bone cement, bio-cement, a flexible titanium structure, surgical sutures, osseo-integration, tissue integration, titanium pins, and/or surface geometry. Or the mechanical stimulation component may not be fixed into position with respect to the auditory tissue but instead utilize a hydro-drive arrangement to communicate the vibrations to the cerebral tissue.

### BRIEF DESCRIPTION OF THE DRAWINGS

FIG. 1 shows structures of a typical ear which includes a cochlear implant system.

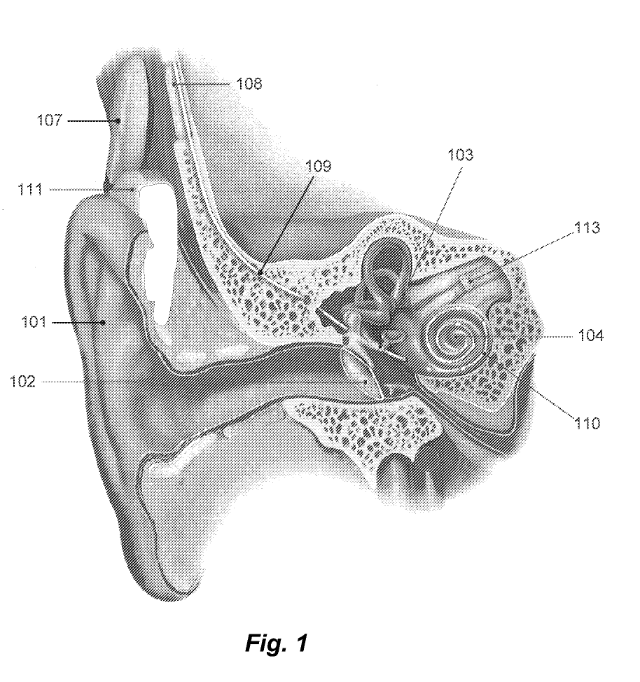

FIG. 2 shows an example of one typical embodiment of a multipath hearing system according to the present invention.

FIG. 3 shows an embodiment which uses a fluid filled catheter to mechanically stimulate auditory tissue.

FIG. 4 shows an embodiment wherein a mechanical stimulation component is integrated into the body of the main implant housing.

FIG. 5 shows a similar embodiment wherein the mechanical stimulation component is at a right angle to the body of the main implant housing.

FIG. 6 shows an example of an embodiment wherein the mechanical stimulation component is located inside a transducer housing that protrudes from the body of the main implant housing.

FIG. 7 shows a similar embodiment wherein the transducer housing is recessed into the body of the main implant housing.

FIG. 8 shows an embodiment which uses the implant magnet as a cooperating part in a mechanical stimulation component.

FIG. 9 shows an embodiment which has a single cable emerging from the main implant housing.

FIG. 10 shows an embodiment of a mechanical stimulation component which is fixed to bone tissue by screws.

### DETAILED DESCRIPTION OF SPECIFIC EMBODIMENTS

Embodiments of the present invention are directed to a prosthetic hearing system that provides multi-path stimulation of the patient auditory system. A mechanical stimulation component applies mechanical stimulation signals to cerebral tissue such as the dura mater, cerebrospinal fluid, vestibular structures, etc. using multiple separate mechanical stimulation channels. And an electrical stimulation component provides electrical stimulation of auditory neural tissue of the patient user.

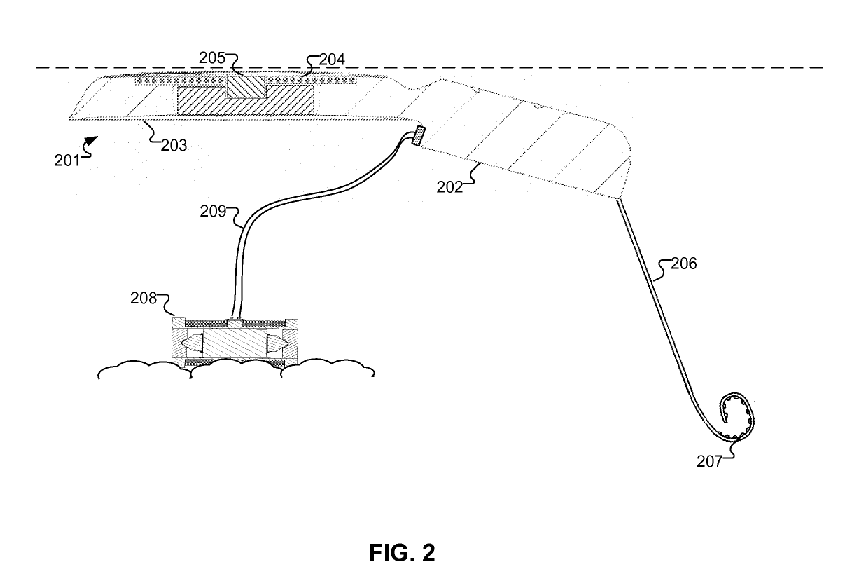

FIG. 2 shows elements of a multipath stimulation hearing prosthesis system according to one embodiment in which a main implant housing 201 includes a electrical stimulation component 202 and an implant coil housing 203 having an implant receiver coil 204 for receiving a transcutaneous implant signal from a corresponding external transmitter coil to provide electrical stimulation of auditory neural tissue of the patient user. A mechanical stimulation component 208 is implanted outside the main implant housing 201 and applies multiple mechanical stimulation signals to cerebral tissue using multiple separate mechanical stimulation channels. The main implant housing 201 may be constructed of a biocompatible material such as medical grade titanium, ceramic, silicone, and acrylic.

For example, the different mechanical stimulation signals and mechanical stimulation channels may stimulate different specific cerebral tissues and locations, including without limitation the dura mater, cerebrospinal fluid, inner ear tissue such as vestibular tissue, and/or bone tissue such as a temporal bone within the middle ear or skull bone. The main implant housing 201 may be fixed to skull bone of the patient user so as to provide mechanical stimulation by bone conduction to the auditory system. At the same time, the mechanical stimulation component 208 may be a floating mass transducer (FMT) as shown in FIG. 2 which provides another mechanical stimulation signal through a separate mechanical stimulation channel to the dura mater by inertial vibration of the FMT. In fact, the mechanical stimulation developed by a mechanical stimulation component 208 such as the one shown in FIG. 2 which is based on an FMT, can be quite sufficiently strong to support multiple mechanical stimulation channels as described herein. The mechanical vibrations specifically may have a displacement less than or equal to 100 microns peak to peak. A fuller discussion of using FMTs for bone conduction mechanical stimulation is provided in U.S. Patent Publication 2007191673, which is incorporated herein by reference.

Besides a floating mass transducer (FMT, in other embodiments, the mechanical stimulation component 208 may be a vibrating element transducer, a balanced armature transducer, an inertial drive transducer, a rotating transducer, a rotational magnet and armature transducer, or a vibrating magnet with an associated coil (e.g., with a secondary magnet element that is repelled and attracted to a remotely located first vibrating magnet element). The mechanical stimulation component 208 may be connected to the main implant housing 201 by a disconnectable cable 209 which carries one or more of the mechanical stimulation signals to the mechanical stimulation component 208.

The coil housing 203 also contains an implant magnet 205 in a mechanically stable position for magnetic interaction with a corresponding external magnet to hold an external data transmitting coil in proper position over the receiver coil 204. The electrical stimulation component 202 includes an electrode carrier 206 having multiple stimulation electrodes 207 which is inserted into the cochlea to provide electrical stimulation of auditory neural tissue such as the cochlear nerve and/or the brainstem. There may also be a secondary electrical ground unit for the stimulation electrodes 207. The electrical stimulation component 202 may be mechanically isolated from the vibrations of the mechanical stimulation component 208. The electrical stimulation component 202 may provide one or more of the mechanical stimulation channels. For example, a mechanical stimulation signal may be applied to and developed within the cochlea by the electrode carrier 206.

The implant housing 201 also may include an implant signal processor for processing the implant signal, which may be generated by a sound sensing arrangement using an external processor together with a microphone arrangement such as an external omni-directional microphone, multiple external sensing microphones, or an implanted microphone for sensing sound. In one specific embodiment, the implant signal processor processes the implant signal based on its frequency content such that a first band of frequencies is associated with the electrical stimulation component 202, and a second band of frequencies is associated with the mechanical stimulation component 208. For example, the second band of frequencies for the mechanical stimulation component 208 may possess a peak resonance audio frequency between 0.25 and 3.5 kHz.

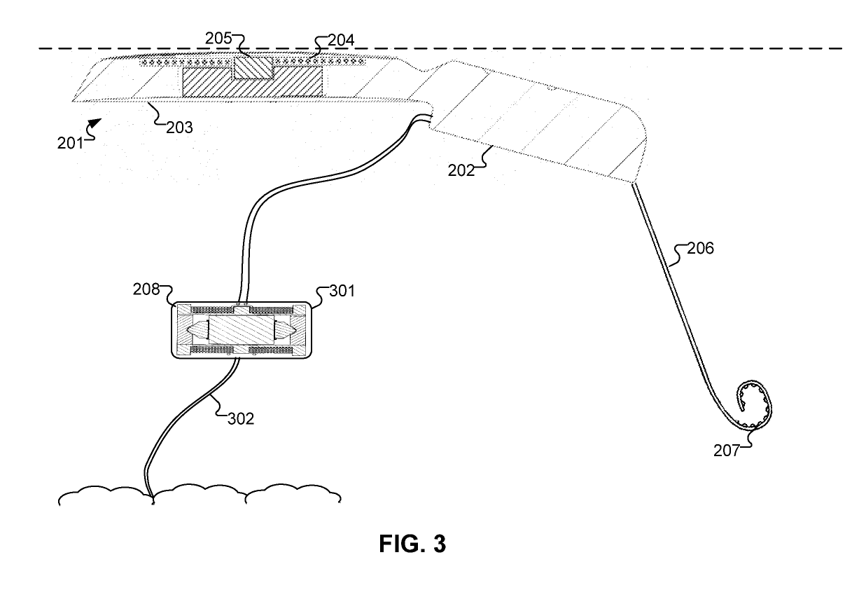

FIG. 3 shows another embodiment in which the mechanical stimulation component 208 is enclosed in a fluid filled transducer housing 301 which is coupled to a fluid-filled catheter tube 302 for coupling the vibration from the mechanical stimulation component 208 to the cerebral tissue. In such an embodiment, additional mechanical stimulation channels may be provided by the transducer housing 301 (e.g., via bone conduction), by the main implant housing 201, and/or by the electrode carrier 206 to develop one or more additional mechanical stimulation signals at additional cerebral tissue locations.

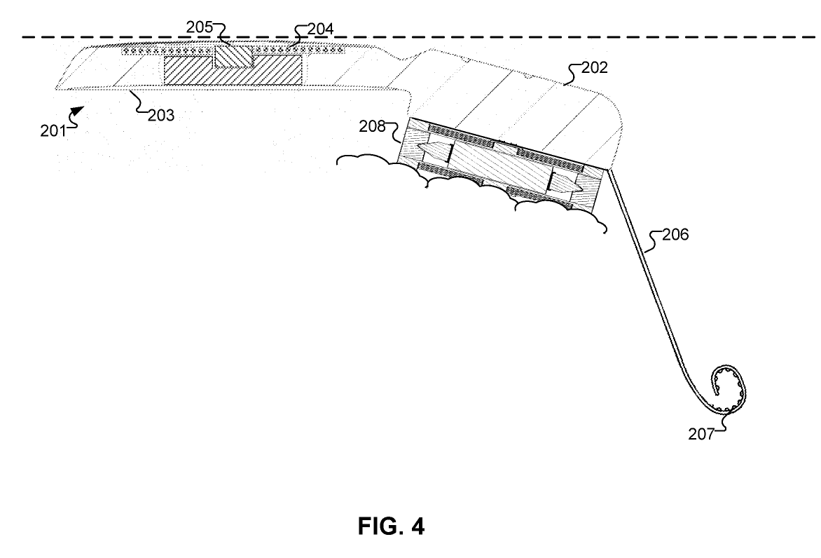

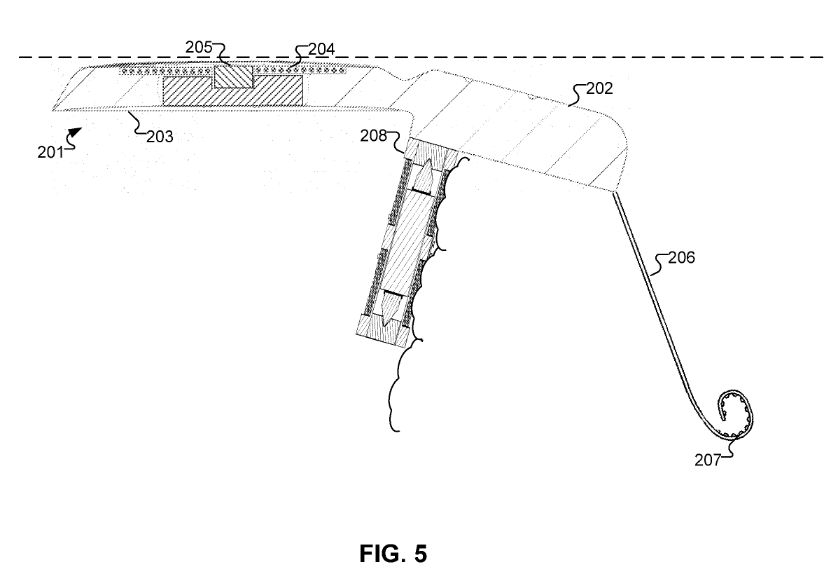

FIG. 4 shows an embodiment for use in implant geometries where the main implant housing 201 is located relatively near to the cerebral tissue to be stimulated by the mechanical stimulation component 208, which is integrated into body of the main implant housing 201. In this specific case, it is integrated into the housing for the electrical stimulation component 202. FIG. 5 shows a similar embodiment for a different implant geometry where the mechanical stimulation component is integrated at a right angle into the main implant housing 201. Again multiple mechanical stimulation channels may be provided by one or more of the mechanical stimulation component 208 (here, an FMT) the main implant housing 201, the housing of the electrical stimulation component 202, and/or the electrode carrier 206 to one or more cerebral tissue locations such as the dura mater, cerebrospinal fluid, inner ear tissue such as vestibular tissue, and/or bone tissue such as a temporal bone within the middle ear or skull bone.

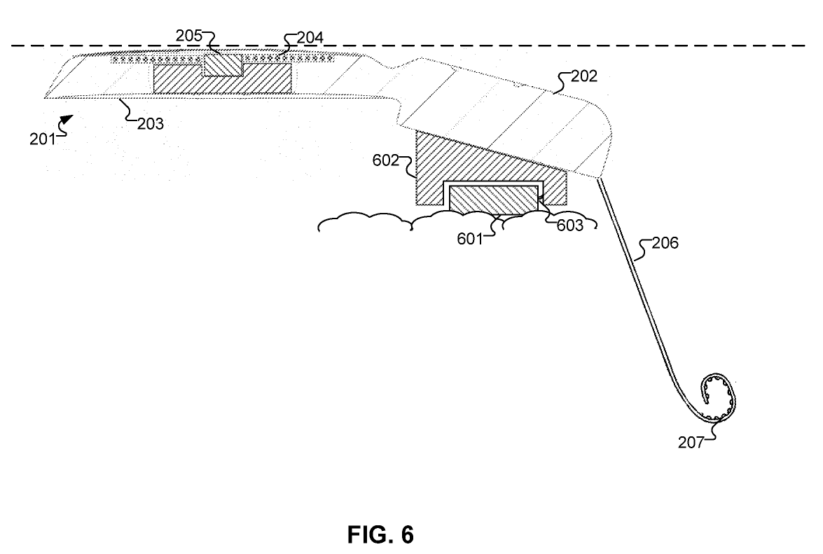

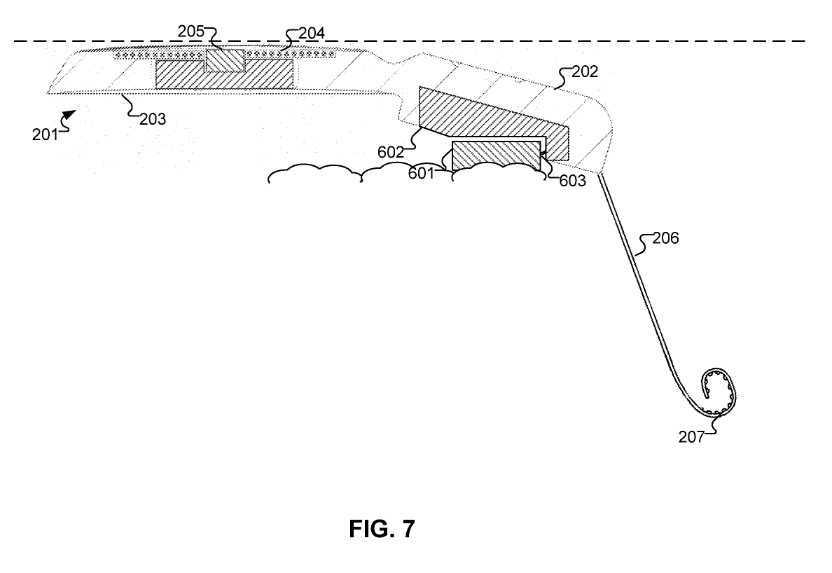

In some embodiments, as shown in FIG. 6, a mechanical stimulation component 601 may be located within a transducer housing 602 that protrudes out from the main implant housing 201. In the embodiment shown, the mechanical stimulation component 601 is coupled to signal processing circuitry within the main implant housing 201 by a connector cable 603, which may be hard wired or a disconnectable arrangement. FIG. 7 shows a related embodiment where the transducer housing 602 is more recessed into the main implant housing 201.

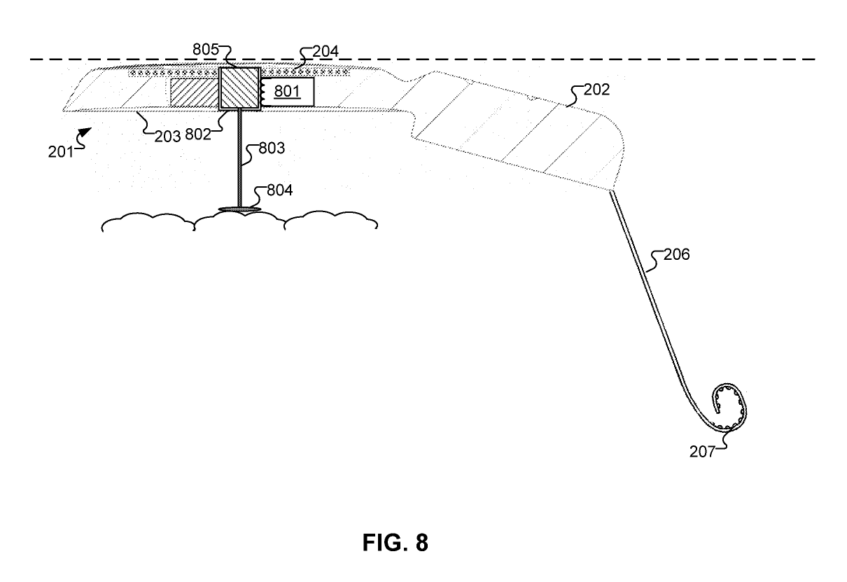

FIG. 8 shows an example of an embodiment where the implant magnet fulfills multiple functions. The implant magnet 805 is suspended in a fluid filled cavity 802 where it serves to hold an external component in an appropriate position and increases the magnetic flux for transcutaneous signal and energy transfer from the external signal coil to the implant receiver coil 204. In addition, the implant magnet 805 is also functionally coupled to operate as a part of the mechanical stimulation component. In the case shown, a magnetic coupling winding 801 cooperates with the implant magnet 805 to displace and vibrate a coupling rod 803 which in turn drives a cup-shaped transducer plate 804 that stimulates the auditory tissue, e.g., the dura mater tissue.

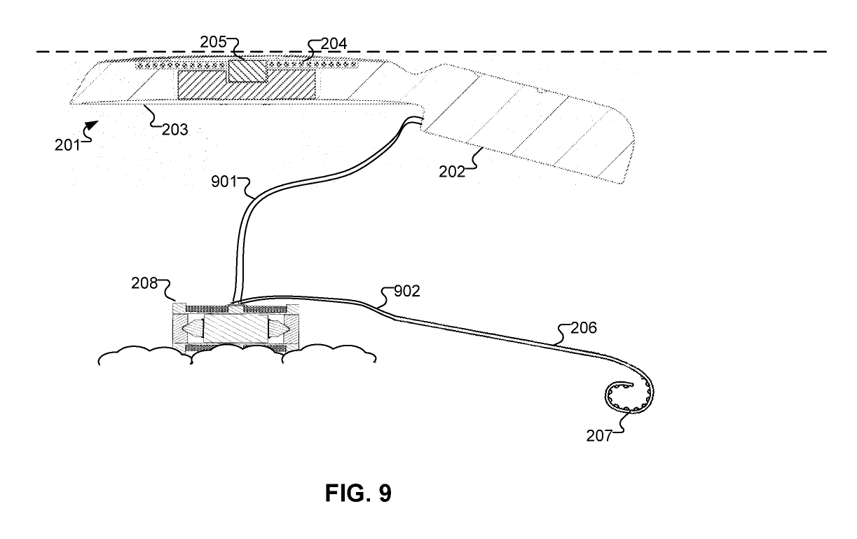

FIG. 9 shows an alternative embodiment wherein a single stimulation cable is connected to the main implant housing 201 and providing the mechanical stimulation signal to mechanical stimulation component 208, after which a continuation cable 902 connects separately to the stimulation electrode 206.

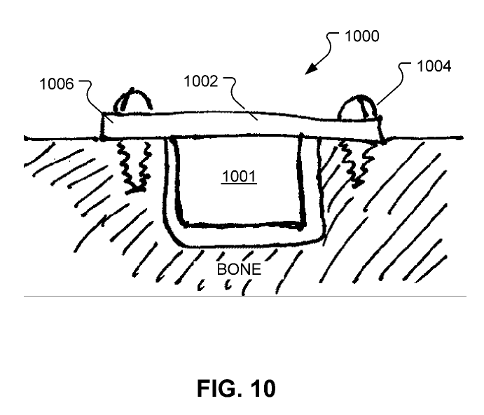

FIG. 10 shows an example of a mechanical stimulation component 1000 that includes an inertial drive transducer 1001 which is fixed into position with respect to underlying bone tissue by one or more screws 1004 through corresponding screw holes 1006 in a transducer coupling plate 1002. In addition or alternatively, a mechanical stimulation component 208 may be fixed into position with respect to the cerebral tissue by a silicone elastomer membrane, bone cement, bio-cement, a flexible titanium structure, surgical sutures, osseo-integration, tissue integration, titanium pins, and/or surface geometry. Or the mechanical stimulation component 208 may not be fixed into position with respect to the auditory tissue but instead utilize a hydro-drive arrangement to communicate the vibrations to the auditory tissue.

In any of the above embodiments, the electrical stimulation component 202 and the mechanical stimulation component 208 may be synchronously operable to operate in parallel at the same time and/or alternately operable to operate serially one at the same time. The implant housing 201 may include an implanted sensing microphone connected to the main implant housing for monitoring performance of the mechanical stimulation component. Similarly, there may be a performance monitor for monitoring and assessing performance of the mechanical stimulation component. The electrical stimulation component may be mechanically isolated from the vibrations of the mechanical stimulation component.

Although various exemplary embodiments of the invention have been disclosed, it should be apparent to those skilled in the art that various changes and modifications can be made which will achieve some of the advantages of the invention without departing from the true scope of the invention. For example, the entire prosthetic hearing system may be implantable within a patient. Some embodiments may include a hybrid operating mode which communicates with external devices such as an external signal processor, diagnostic devices, and/or an external power supply arrangement.

What is claimed is:

1. A prosthetic hearing system, comprising:
a mechanical stimulation component for applying a plurality of mechanical stimulation signals to cerebral tissue of a patient user using a plurality of separate mechanical stimulation channels; and
an electrical stimulation component for electrical stimulation of auditory neural tissue of the patient user.

2. A system according to claim 1, wherein the mechanical stimulation component includes a floating mass transducer for creating inertial vibration of the cerebral tissue.

3. A system according to claim 1, where the mechanical stimulation component includes a vibrating element transducer, a balanced armature transducer, an inertial drive transducer, or a rotating transducer.

4. A system according to claim 1, wherein the mechanical stimulation component includes a rotational magnet and armature transducer.

5. A system according to claim 1, wherein the mechanical stimulation component includes a vibrating magnet with an associated coil.

6. A system according to claim 5, wherein the vibrating magnet includes a secondary magnet component that responds to a remotely located primary vibrating magnet component.

7. A system according to claim 1, wherein the mechanical stimulation component includes a vibrating plate in contact with the cerebral tissue.

8. A system according to claim 1, further comprising:
a main implant housing containing receiver circuitry for receiving and processing an externally generated implant signal.

9. A system according to claim 8, wherein the main implant housing is directly coupled to the cerebral tissue to provide at least one of the mechanical stimulation channels.

10. A system according to claim 9, wherein the main implant housing provides a plurality of mechanical stimulation channels.

11. A system according to claim 9, wherein the mechanical stimulation component includes a vibrating coupling rod functionally coupled to mechanically stimulate the cerebral tissue.

12. A system according to claim 8, wherein the main implant housing further contains the electrical stimulation component and the mechanical stimulation component.

13. A system according to claim 12, wherein the electrical stimulation component provides at least one of the mechanical stimulation channels.

14. A system according to claim 1, further comprising:
an electrical module housing associated with the electrical stimulation component; and
a mechanical module housing associated with the mechanical stimulation component and separate from the electrical module housing.

15. A system according to claim 14, wherein each module housing provides at least one of the mechanical stimulation channels.

16. A system according to claim 1, wherein at least one of the mechanical stimulation channels is provided by a fluid-filled catheter tube.

17. A system according to claim 1, wherein the electrical stimulation component provides at least one of the mechanical stimulation channels.

18. A system according to claim 17, wherein the electrical stimulation component provides a plurality of mechanical stimulation channels.

19. A system according to claim 1, wherein the cerebral tissue of one of the mechanical stimulation channels includes dura mater tissue.

20. A system according to claim 1, wherein the cerebral tissue of one of the mechanical stimulation channels includes cerebrospinal fluid (CSF).

21. A system according to claim 1, wherein the cerebral tissue of one of the mechanical stimulation channels includes inner ear tissue.

22. A system according to claim 21, wherein the inner ear tissue includes vestibular tissue.

23. A system according to claim 1, wherein the cerebral tissue of one of the mechanical stimulation channels includes bone tissue.

24. A system according to claim 23, wherein the bone tissue includes temporal bone tissue.

25. A system according to claim 23, wherein the bone tissue includes skull bone tissue.

26. A system according to claim 1, wherein the auditory neural tissue includes cochlear nerve tissue.

27. A system according to claim 1, wherein the auditory neural tissue includes brainstem tissue.

28. A system according to claim 1, wherein the mechanical stimulation module is not fixed into position with respect to the cerebral tissue but instead utilizes a hydro-drive arrangement for mechanically stimulating the cerebral tissue.
